(19) United States
(12) Patent Application Publication
(10) Pub. No.: US 2025/0288720 A1
(43) Pub. Date: Sep. 18, 2025

(54) COATING FOR A BIORESORBABLE SCAFFOLD

(71) Applicant: Shanghai Bioresorbable Scaffold Technology Co. Ltd., China (Shanghai) Pilot Free Trade Zone (CN)

(72) Inventors: Ty Tiefeng HU, China (Shanghai) Pilot Free Trade Zone (CN); Wang RONG, China (Shanghai) Pilot Free Trade Zone (CN)

(21) Appl. No.: 18/607,819

(22) Filed: Mar. 18, 2024

(51) Int. Cl. A61L 27/34; A61L 27/54; A61L 27/58

(52) U.S. Cl. CPC A61L 27/34; A61L 27/54; A61L 27/58; A61L 2300/426; A61L 2300/606; A61L 2400/18; A61L 2420/02; A61L 2420/08

(57) ABSTRACT

The present invention relates to a coating for a bioresorbable scaffold, comprising a degradation control layer comprising a durable hydrophobic polymer and a biodegradable hydrolytic polymer; a bioresorbable scaffold comprising a stent body and such a degradation control layer; a bioresorbable scaffold comprising this coating; and a method of coating a stent body to make a bioresorbable scaffold.

### FIELD OF INVENTION

The present invention relates to a coating for a bioresorbable scaffold, such as bioresorbable polylactic acid scaffolds. The invention also relates to a bioresorbable scaffold comprising this coating and a method for coating a stent body to make the bioresorbable scaffold.

### BACKGROUND

Stents expand narrowed blood vessels due to atherosclerosis or other reasons and provide support, thereby relieving vessel stenosis and improving ischemic symptoms. They could be used to treat coronary, carotid, peripheral or other artery stenosis. They are also used to treat aneurysms. The process of stent implantation is an interventional procedure, typically achieved through self-expansion or balloon dilation to position and release the stent.

Currently, the stents used for the treatment of coronary and peripheral blood vessels are metal drug-eluting stents, which consist of a polymer drug coating and a non-degradable metal scaffold (usually made of 316 stainless steel alloy or L506 cobalt-chromium alloy). However, the core issue with stents at present is that the long-term stimulation of the stent in the blood vessel may cause neointima hyperplasia and subsequently accelerate neoatherosclerosis. As a result, there is a 3% annual occurrence of stent failure after implantation.

The concept of bioresorbable scaffolds (abbreviated as “BRS” below) aims to address the problem of stent failure caused by the long-term presence of the stent in the blood vessel. Commercially available BRS mainly includes polymer-based scaffolds, such as poly-lactic acid, and metal-based degradable stents, such as magnesium alloys. However, current commercially available BRS have not demonstrated superior effectiveness to drug-eluting stents in clinical study, and even exhibit significantly higher rates of scaffold thrombosis and in-stent restenosis. The core mechanism behind the increased occurrence of scaffold thrombosis within the polymer-based BRS is the unstable degradation of the polymer, which leads to scaffold fracture and prolapse. Additionally, polymer-based BRS is more prone to activate platelets compared to metal stents.

When the inventors reviewed existing BRS technology, the inventors found that optimizing the scaffold degradation process is a key factor in further improving its performance. A more stable degradation process can avoid uneven degradation of the scaffold, thereby reducing scaffold fracture and prolapse, and the occurrence of scaffold thrombosis. A longer degradation period can provide targeted vessel with more sufficient remodeling time while reducing the occurrence of in-scaffold restenosis caused by explosive degradation and subsequent vascular inflammation reaction.

Currently, strategies based on optimizing the scaffold material to achieve control over the degradation period have reached a bottleneck. It's difficult to achieve ideal control over the degradation period before breakthroughs in material technology occur. Therefore, it is important to search for other ways to optimize the degradation period and improve core performance of the scaffold, such as thrombosis resistance and endothelialization.

### SUMMARY OF THE INVENTION

In the first aspect, the present invention provides a coating for a bioresorbable scaffold, comprising a degradation control layer comprising a durable hydrophobic polymer and a biodegradable hydrolytic polymer. Advantageously, the degradation control layer effectively regulates the degradation rate of the scaffold matrix, the hydrophobic layer maintains a smooth surface of the stent. As a result, controlled degradation of the scaffold matrix is achieved, reducing the likelihood of thrombosis, accelerating endothelialization, and preventing in-stent restenosis. It will be appreciated that polymers used in the coating are biocompatible to avoid causing harm to the patient. The coating may be alternatively called a “BRS coating” or “stent coating”.

In some embodiments, the hydrophobic polymer may comprise at least one polymer selected from a group consisting of: acrylics, amides, imides, carbonates, dienes, esters, ethers, fluorocarbons, olefins, styrenes vinyl acetals, vinyl and vinylidene chlorides, vinyl esters, vinyl ethers, vinyl ketones, vinylpyridine, vinypyrrolidone, poly(vinylidene chloride), polyvinylidene fluoride (PVDF), polyvinylidene fluoride-co-hexafluoropropylene (PVDF-HFP), styrene-butadiene-styrene copolymer (SBS), polyurethane (PU), polymethyl methacrylate (PMMA), and polybutyl methacrylate (PBMA), poly(N-vinylpyrrolidone), poly(vinylpyridine), poly(vinylpyrrolidone), polydimethylsiloxane (PDMS), poly-xylene (PX), and combinations thereof. These polymers from the stent material from interacting with water and other substances in blood, and help to maintain the integrity of the stent and control the degradation.

In some embodiments, the hydrolytic polymer comprises at least one polymer selected from the group comprising: polylactic acid (PLA), poly-L-lactic acid (PLLA), poly-D-lactic acid (PDLA), poly-DL-lactic acid (PDLLA), poly(lactic-co-glycolic acid) (PLGA), poly(lactic-co-glycolic) acid, Dextron, nylon-2-nylon-6, PHBV polymer (Poly(3-hydroxybutyrate-co-3-hydroxyvalerate)), poly-L-proline ester-hexyl lactate copolymer, polyglycolic acid (PGA), polyhydroxy butyrate (PHB), polycaprolactone, polyglycolide, polylactide, chitosan, hyaluronic acid, poly(2-hydroxyethyl-methacrylate), poly(L-lactide), polyethylene glycol, chitosan, and combinations thereof. In particular, the hydrolytic polymer may comprise at least one polymer of poly-L-lactic acid, poly-caprolactone, and poly-L-proline ester-hexyl lactate copolymer, polyglycolic acid and/or polylactic acid. The hydrolytic polymer (alternatively a “hydrolysable” polymer) may be biocompatible, to reduce the likelihood of triggering an immune response in the body after insertion. Thickness of the degradation control layer may be in the range of 200 nm and 20,000 nm. This range of stent wall thickness enables a broad range of applications. The hydrolytic polymer may be in the range between 0% to 50% by mass fraction. These ranges have been shown to provide consistent degradation of the coating. Different hydrolytic polymers may be use in different proportions to produce desired bioresorption rates.

In some embodiments, the degradation control layer may comprise a medicament, so that the medicament is released as the layer is degraded, thus providing additional therapeutic effects.

In some embodiments, the coating may further comprise a drug layer comprising a drug. The drug may comprise an antiproliferative drug and/or an immunosuppressant. An antiproliferative drug can inhibit intimal hyperplasia for better clinical outcome. For example, the immunosuppressant may comprise at least one compound selected from the group comprising sirolimus, everolimus, zotarolimus, tacrolimus, paclitaxel, and combinatios thereof. The drug may be deposited in a layer of thickness in the range of 500 nm and 10,000 nm. The drug amount per unit length of the coating may be in the range of 3 μg/mm to 25 μg/mm. A ratio of the drug to a drug carrier may be in the range of 3:1 to 1:10. These ranges have shown reliable drug release rates. The drug carrier may be at least one hydrolytic (or hydrolyzable) polymer selected from a group consisting of polylactic acid (PLA), poly-L-lactic acid (PLLA), poly-D-lactic acid (PDLA), poly-DL-lactic acid (PDLLA), poly(lactic-co-glycolic acid) (PLGA), poly(lactic-co-glycolic) acid, Dextron, nylon-2-nylon-6, PHBV polymer (Poly(3-hydroxybutyrate-co-3-hydroxyvalerate)), polyglycolic acid (PGA), polyhydroxy butyrate (PHB), polycaprolactone, polyglycolide, polylactide, chitosan, hyaluronic acid, poly(2-hydroxyethyl-methacrylate), poly(L-lactide), polyethylene glycol, chitosan, and combinations thereof. Adjusting the ratio of the drug to the drug carrier in the drug layer enables controlled release of the drug. The drug can provide additional medical properties, such as anti-thrombosis function.

In some embodiments, thickness of the hydrophobic layer is in the range of 200 nm to 10,000 um.

In a second aspect, the invention provides a bioresorbable scaffold comprising a stent body and a coating described above. The stent body may comprise at least one hydrolytic polymer comprising polylactic acid and/or polyglycolic acid. This enables the stent body to be biodegradable and absorbed by the body. It will be appreciated that the stent body comprises a biocompatible materials to avoid causing harm to the patient.

In some embodiments, the stent may comprise a drug layer. The drug layer may comprise an antiproliferative drug that can inhibit intimal hyperplasia for better clinical outcome.

In a third aspect, the invention provides a method of coating a stent body to make a bioresorbable scaffold, comprising: depositing a degradation control layer on a stent body, wherein the degradation control layer comprises a durable hydrophobic polymer and a biodegradable hydrolytic polymer. This method creates a bioresorbable scaffold that has controllable degradation rates, for potential drug release and stent monitoring.

In some embodiments, the hydrophobic layer may form a discontinuous layer around the degradation control layer. This discontinuous layer allows pores or crevices for water to enter and interact with the degradable layer, thus triggering the biodegradation process. The discontinuous layer could also refer to asymmetric coating of the vascular stent, such as leaving a lengthwise section of the stent uncoated. The degradation control layer may comprise a drug.

In some embodiments, the method may further comprise depositing a drug layer comprising a medicament dissolved in a solvent. The solvent may be an organic solvent. This drug layer confers additional medical properties to the resulting vascular stent. The drug layer may comprise an antiproliferative drug and/or an immunosuppressant. An antiproliferative drug can inhibit intimal hyperplasia for better clinical outcome. An immunosuppressant such as sirolimus, everolimus, zotarolimus, tacrolimus, and paclitaxel, can reduce the likelihood of the patient generating an immune response against the stent.

In some embodiments, the depositing may comprise ultrasonic spray coating. Spray coating could create a discontinuous layer efficiently.

In some embodiments, the degradation control layer and/or the drug layer may comprise a polymer dissolved in a volatile organic solvent at a concentration between 0.5 mg/ml and 50 mg/ml by weight prior to depositing.

In some embodiments, the stent body may comprise at least one hydrolytic polymer comprising polylactic acid and/or polyglycolic acid. This enables the stent body and bioresorbable scaffold to be biodegraded.

### BRIEF DESCRIPTION OF THE FIGURES

It will be convenient to further describe the present invention with respect to the accompanying drawings that illustrate possible arrangements of the invention. Other arrangements of the invention are possible and consequently, the particularity of the accompanying drawings is not to be understood as superseding the generality of the preceding description of the invention.

FIG. 1 is a schematic diagram of a multi-functional composite coating for a bioresorbable vascular stent.

FIG. 2 is a schematic diagram of a hollow ultrasonic spraying process.

FIG. 3 is a schematic diagram of an axial ultrasonic spraying process.

FIG. 4A shows bioresorbable scaffolds (abbreviated as “BRS” below) degradation curves for different coating in an in vitro simulation experiment, expressed as the number average molecular weight Mn.

FIG. 4B shows graphs of degradation curves of a novel polymer BRS of the present invention and a first generation BRS in an animal model, in terms of number averages molecular weight Mn and weight averaged molecular weight Mw.

FIG. 5 show optical coherence tomography (OCT) and fluorescence images of a novel polymer BRS coating of the present invention 50 vs first generation BRS 51. The new polymer coating shows better coverage of endothelial cells on the stent.

FIGS. 6A-D are photographs of various bioresorbable scaffolds. Three bioresorbable scaffolds are shown side-by-side for direct comparison (A)-a novel polymer BRS coating of the present invention 61 (B), a metal drug-eluting stent (DES) 62 (C), a first generation BRS coating 63 (D).

FIG. 7 are OCT images of a novel polymer BRS coating of the present invention and a first generation BRS at post-procedure, 1-month follow up (1M FU), 3-month follow up (3M FU), 6-month follow up (6M FU), and 12-month follow up (12M FU).

FIG. 8 are histopathological sections of a stent with the new polymer coating of the present invention at 3 months (3M) and 6 months (6M) follow up at different magnifications (4×, 10×, 20×).

### DETAILED DESCRIPTION

### Problems With Existing BRS

The shortcomings of current bioresorbable scaffold in clinical applications are mainly as follows:

### a) Degradation Kinetics of BRS

The ideal degradation curve for BRS should be uniform and stable, which can minimize inflammatory reactions during degradation and prevent the occurrence of weak points that can lead to collapse or fracture of the scaffold.
However, current commercially available BRS have not been able to achieve the ideal degradation curve. For example, since polymer-based BRS such as poly-lactic acid have a rapid decrease in molecular weight between 6-9 months, and are then phagocytized by macrophages, this is currently considered the time period when scaffold restenosis and thrombosis occur the most.
Existing technology improvements mainly focus on poly-lactic acid and degradable metal tubes, although many efforts have been made (ultra-high molecular weight poly-lactic acid, poly-lactic acid modification, alloy formulation adjustment), precise control of the degradation curve of the tube is still not achievable at present.

### b) Scaffold Discontinuity and Prolapse

Scaffold discontinuity is the most significant characteristic that distinguishes biodegradable scaffolds from metal stents. Due to various factors, such as the morphology of the lesion tissue and stress, there may be differences in the degradation process of the scaffold at different sites, resulting in scaffold structural discontinuity and fracture.

When scaffold discontinuity occurs, with scaffold struts not completely covered by endothelium, the scaffold may detach into the lumen, resulting in what is known as scaffold prolapse. The exposed struts in the bloodstream can lead to platelet adhesion on the scaffold surface, activating the platelets and subsequently inducing the formation of scaffold thrombosis.

Previous studies have shown that scaffold prolapse occurs in more than 10% of patients implanted with poly-lactic acid BRS, which is significantly correlated with late scaffold thrombosis. More than 40% of patients with scaffold thrombosis exhibit scaffold discontinuity. Therefore, effectively reducing scaffold discontinuity and prolapse is one of the key factors in addressing long-term adverse events of biodegradable scaffolds. However, current material science technology still cannot guarantee uniform scaffold degradation, and cannot guarantee complete endothelial repair before loss of scaffold mechanical performance occurs.

### c) Delayed Endothelial Healing of BRS

Complete endothelial coverage of the scaffold surface is one of the key factors in preventing scaffold thrombosis. For BRS, endothelial healing also means a significant reduction in the occurrence of scaffold prolapse.

However, currently, polymer-based biodegradable scaffolds exhibit delayed endothelial healing compared to metal stents, and this phenomenon has been observed in animal experiments and clinical studies. The delayed endothelial healing of biodegradable scaffolds is closely related to two factors. First, in order to maintain radial support, the strut thickness of biodegradable scaffolds is twice that of non-degradable metal stents, and it has been proven that the strut thickness is related to the speed of endothelial healing. Furthermore, the characteristics of poly-lactic acid scaffold can also cause delayed endothelial healing. Previous studies have shown that the endothelial healing rate of poly-lactic acid coatings is significantly lower than that of metal stents. Additionally, the inflammatory response caused by scaffold degradation may also affect endothelial function. The inventors' study in rabbits confirmed this, using poly-lactic acid scaffolds and metal scaffolds with the same strut thickness and drug dosage. The endothelial healing rate of the poly-lactic acid scaffold was still significantly lower than that of the metal stents.

Reducing the strut thickness and improving the endothelial-friendliness of the scaffold material are obviously effective methods to enhance the endothelial healing rate of BRS. However, currently, polymer-based materials still cannot match metal materials in terms of mechanical performance. At the same time, the improvement of polymer biocompatibility is still in the laboratory stage, and its impact on the mechanical performance of the tube cannot meet the requirements of mass production. Therefore, different optimization strategies are needed to enhance the endothelial healing rate of BRS.

### d) Antithrombotic Performance of Polymer Materials

Polymer-based materials causing thrombosis is another important factor contributing to the increased occurrence of thrombosis in polymer-based BRS. Previous studies have shown that the thrombosis rate of polymer-based BRS is significantly higher than that of metal stents after implantation, not only during the late degradation process. Through in vitro experiments, researchers have confirmed that polymer-based BRS is more likely to induce platelet adhesion and neutrophil aggregation compared to metal stents, which are important mechanisms of platelet activation. Currently, almost all polymer-based scaffolds use poly-lactic acid as the scaffold material. It is challenging to enhance antithrombotic performance while ensuring mechanical properties. Currently, there is no polymer-based scaffold that has been proven to achieve the same level of antithrombotic performance as metal stents.

### Multifunctional Protective Coating Technology Applied to BRS

The present invention provides a coating for a bioresorbable scaffold and a bioresorbable scaffold comprising this coating and a stent body. This coating comprises a degradation control layer comprising a durable hydrophobic polymer and a biodegradable hydrolytic polymer. The degradation control layer is composed of durable polymers with low water absorption and hydrolytic polymers with high absorption rates. The hydrophobic layer is made up of durable polymers with low water absorption. The coating also comprises a drug layer that consists of drugs and drug carriers.

The multifunctional coating can achieve the following functions:
a) precise control of the degradation process of the BRS:
   the extremely low water absorption of this durable polymer can effectively block the exchange of liquid between the stent substrate and the external environment, delaying the hydrolysis process and thereby slowing the degradation process of the polymer-based scaffold. The degradation process can be further delayed by increasing the thickness of the coating. Additionally, the durable polymer can be co-blended with a highly water-absorbable biodegradable polylactic acid material to accelerate hydrolysis and thus the degradation process by increasing the proportion of polylactic acid in the blend. The degradation control layer may contain a medicament, so the rate of release of the medicament can be controlled.
b) superior anti-thrombogenic performance of the stent surface: the extremely low coefficient of friction of this durable polymer results in a very smooth surface of the coating formed on the stent surface, effectively reducing platelet adhesion and hence reducing thrombus formation.
c) acceleration of endothelial healing: the excellent biocompatibility and precise control of the degradation process of the stent can reduce adverse stimulation of the vascular endothelium during the degradation process, effectively accelerate endothelial coverage and maintain endothelial cell barrier function.
d) avoidance of scaffold prolapse during the degradation process: during the degradation process of the scaffold, the durable polymer coating can form a complete protective sheath (protective net) on the outer surface of the stent, allowing only small molecule degradation products or micro/nano-scale degradation products to pass through, thereby avoiding scaffold prolapse due to incomplete coverage of scaffold struts by endothelium during degradation., which is one of the most common mechanism of scaffold thrombosis. The durable polymer coating is long-lasting and provides long-term protection.
e) effective inhibition of neointimal hyperplasia and in-stent restenosis can be achieved through controlled drug release from the drug layer.

The conventional idea for a stent coating is to use a coating material that can also be absorbed and its function is only to carry antiproliferative drug to release of inhibit neointimal hyperplasia after stent implantation. In contrast, the inventors have created stents using durable polymers coating to achieve precise control of the scaffold degradation process, while optimizing its anti-thrombogenic and endothelialization properties and solving the problem of scaffold prolapse.

In one embodiment of the invention, the durable polymer comprises polyvinylidene fluoride-co-hexafluoropropylene (PVDF-HFP). This polymer has extremely low water absorption, a low coefficient of friction, and good biocompatibility. Other durable polymers that can achieve similar functions include polyvinylidene fluoride (PVDF), styrene-butadiene-styrene copolymer (SBS), polyurethane (PU), polymethyl methacrylate (PMMA), polybutyl methacrylate (PBMA), polydimethylsiloxane (PDMS), and poly-xylene (PX), etc.

In another embodiment of the invention, the stent comprises the same durable polymer as mentioned above, such as PVDF or PVDF-HFP. By adjusting the solvent type, solvent concentration, and annealing process after spraying, different mesh sizes of coating morphology can be obtained to regulate the ability of the coating to block water and thereby regulate the degradation rate. Similarly, commonly used drugs for vascular stents, such as sirolimus, everolimus, zotarolimus, tacrolimus, and paclitaxel, can be added to the coating, either as a medicament in the degradation control layer or a drug layer. The release rate of the drugs can be adjusted by a layered approach with different drug ratios in each layer to fully exploit the advantages of the durable polymer as a drug layer and degradation control layer, thereby minimizing the thickness of the drug layer and maximizing the anti-thrombotic effect.

However, in terms of the other important component of the stent, drug layer, all currently available biodegradable stents use biodegradable coatings with single drug loading function. The coating degrades one month after implantation and provides no help in terms of biodegradable stent degradation kinetics, anti-thrombosis, or reduction of stent fractures.

From the current clinical and basic research conclusions, the core advantage of biodegradable stents lies in the restoration of vascular motion function, and complete bioabsorption without residual scaffold is no longer the core requirement for biodegradable stents. Therefore, drug coatings with good biocompatibility can still restore vascular motion function without affecting the hydrolysis of scaffold materials, and a minimal amount of coating material residue does not cause significant long-term inflammatory reactions in blood vessels, avoiding the occurrence of late adverse events caused by long-term stimulation of the vessel wall by foreign bodies.

To address the above problems, the present invention provides a multifunctional composite polymer coating with good biocompatibility applied on the surface of high-polymer bioresorbable stents to achieve the following: (1) a more stable and controllable degradation process of the biodegradable stent scaffold; (2) excellent anti-thrombotic properties of the bioresorbable stent; (4) accelerated endothelialization process to prevent late stent thrombosis caused by strut dislodgement into the lumen after scaffold degradation and fracture.

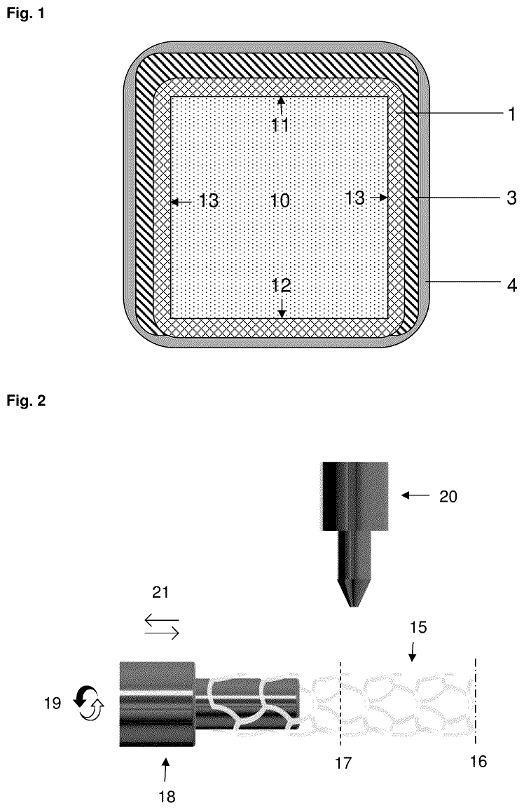

FIG. 1 shows a schematic representation of a cross-section of a bioresorbable stent according to an embodiment of the present invention. The stent comprises a stent body 10, a degradation control layer 1, a hydrophobic layer 3, and optionally a drug layer 4. The thickness of each layer in different cross-sectional directions (11, 12, 13) may be different, to further create precise degradation control of the stent. For example, the hydrophobic layer 3 can be thicker in the direction facing the vascular wall 11 than other directions.

The stent body 10 is a mesh-like structure comprising one or more of the following polymers: polylactic acid, poly-L-lactic acid, poly-caprolactone, and poly-L-proline ester-hexyl lactate copolymer.

The degradation control layer 1 is a mixture of hydrophobic and hydrolytic high polymers. The hydrolytic polymers interact with water to break down over time. For example, the hydrolytic high polymer accounts for 0% to 50% by mass fraction, and is completely applied to the surface of the stent body by ultrasonic spray coating, with a thickness between 200 nm and 20,000 nm.

The hydrolytic high polymers comprise a polymer selected from the group comprising: polylactic acid and its copolymers, including but not limited to polylactic acid (PLA), poly-L-lactic acid (PLLA), poly-D-lactic acid (PDLA), poly-DL-lactic acid (PDLLA), and poly(lactic-co-glycolic acid) (PLGA). These hydrolytic high polymers also have good biocompatibility and hydrolysis characteristics, and can be mixed with the hydrophobic high polymers and dissolved in volatile organic solvents at room temperature. They can then be applied onto the surface of the stent through ultrasonic spraying technology. By adjusting the blending ratio of the hydrolytic high polymers, the swelling period of the stent can be shortened or prolonged, thereby achieving control over the degradation rate of the stent.

The hydrophobic layer 3 may fully encapsulate the degradation control layer 1, or only coat the degradation control layer 1 partially.

FIG. 1 shows a partial coating of the hydrophobic layer 3 over the degradation control layer 1, where the hydrophobic layer is selectively coated on the direction of blood vessel wall 11 and side surface 13 of the degradation control layer 1 by ultrasonic spray coating, without covering the direction of the vascular lumen 12. In other words, there is asymmetric distribution of hydrophobic layer 3. The thickness of the hydrophobic layer 3 is between 200 nm and 10,000 nm. By contrast, the degradation control layer 1 is coated around the full periphery of the stent body 10.

The hydrophobic layer 3 comprises polymers such as polyvinylidene fluoride, poly(vinylidene fluoride-co-hexafluoropropylene), styrene-butadiene-styrene block copolymer, poly(methyl methacrylate), and poly(butyl methacrylate), and combinations thereof. Hydrophobic polymers are not soluble in water. They form a protective layer over the scaffold, and help to maintain the structure of the scaffold.

The hydrophobic polymers suitable for using in the present invention have good biocompatibility, antithrombotic properties, and extremely low water absorption rates. Moreover, they can all dissolve in volatile organic solvents, and can be applied onto the surface of the stent through ultrasonic spraying technology to form a protective layer. This protective layer can effectively reduce thrombus formation during the implantation period and isolate the stent from the surrounding aqueous environment, thereby delaying the degradation rate of the stent by prolonging its swelling period.

The drug layer 4 comprises at least one medicament and at least one drug carrier. FIG. 1 shows a drug layer completely coated on the outer surface of the degradation control layer 1 and the hydrophobic layer 3. The layer may be deposited at a thickness between 500 nm and 10,000 nm by ultrasonic spray coating.

The medicament may comprise sirolimus, tacrolimus, everolimus, zotarolimus, paclitaxel, and others. Such a medicament can inhibit neointimal hyperplasia and inflammation. For example, the medicament content per unit length of the stent is between 3 μg/mm and 25 μg/mm.

The drug carrier comprises at least one hydrolytic high polymer. The hydrolytic high polymer comprises a polymer selected from the group comprising: polylactic acid and its copolymers, including but not limited to polylactic acid (PLA), poly-L-lactic acid (PLLA), poly-D-lactic acid (PDLA), poly-DL-lactic acid (PDLLA), and poly(lactic-co-glycolic acid) (PLGA). The proportion of the scaffold drug content to the drug carrier can be adjusted according to different requirements of the scaffold diameter, the implanted location of the blood vessel, and the degradation rate of the scaffold matrix, etc. For example, the ratio of medicament to drug carrier is between 3:1 and 1:10.

### Method for Preparing a Vascular Stent

The present invention further provides a method for preparing a vascular stent, which comprises the following steps:
1) Shaping the stent body into a mesh-like structure by laser cutting;
2) Dissolving the hydrophobic high-polymer and hydrolytic high-polymer in a volatile organic solvent, and applying the solution to the surface of the stent body by hollow ultrasonic spray coating, followed by drying to form the degradable layer;
3) Dissolving the hydrophobic high-polymer in a volatile organic solvent, and applying the solution to the outer surface of degradable layer by coaxial ultrasonic spray coating, followed by drying to form the hydrophobic layer;
4) Dissolving the medicament and drug carrier in a volatile organic solvent, and applying the solution to the outer surface of the degradable layer and the hydrophobic layer by hollow ultrasonic spray coating, followed by drying to form the drug layer, thereby completing the preparation of the multi-functional composite coating high-polymer biodegradable stent according to the present invention.

In accordance with specific embodiments of the present invention, the volatile organic solvent used in the preparation method of the BRS coating comprises acetone, tetrahydrofuran, dichloromethane, and ethyl acetate, and the concentration of the high-polymer in the volatile organic solvent is between 0.5 mg/mL and 50 mg/mL, based on the weight concentration.

FIG. 2 shows a method of preparing a vascular stent according to another embodiment of the present invention. The stent scaffold 15 is mounted on the hollow spray coating fixture 18 and placed directly under the ultrasonic spray head 20; the length of the hollow spray coating tooling 18 is less than 50% of the length of the scaffold 15, and the scaffold 15 rotates axially 19 with the hollow spray coating tooling 18 while performing reciprocating motion 21 between the distal end 16 and the midpoint 17 of the scaffold; the volatile organic solvent is atomized by the ultrasonic spray head and then sprayed onto the surface of the scaffold 15. When the spray weight reaches 50% of the target weight, the installation direction of the scaffold 15 on the hollow spray coating fixture 18 is changed, and the above steps are repeated to complete the hollow ultrasonic spray coating process.

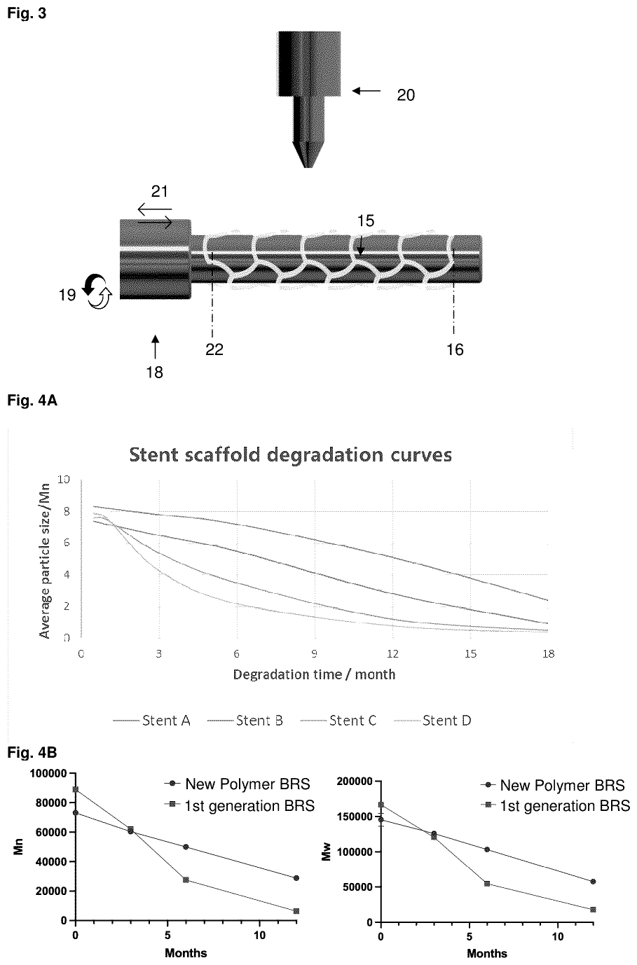

In FIG. 3, the preparation method of a vascular stent comprises a through-axis ultrasonic spraying process: mounting the stent scaffold 15 onto the through-axis spraying fixture and positioning it directly beneath the ultrasonic nozzle 20. The length of the through-axis spraying fixture is greater than the length of the stent scaffold 15, and the stent scaffold rotates axially 19 with the through-axis spraying fixture while performing reciprocating motion 21 between the distal end 16 and the proximal end 22 of the stent scaffold 15; the volatile organic solvent is atomized by the ultrasonic nozzle 20 and sprayed onto the outer surface and lateral surface of the stent scaffold 15 in the direction of the blood vessel wall, while the inner surface of the stent scaffold 15 is not coated due to the blocking of the through-axis fixture, thus achieving directional ultrasonic spraying.

FIG. 4A shows that in in vitro simulated degradation experiments, the degradation control layer provided by the present invention can effectively control the degradation process of the scaffold substrate. FIG. 4B shows that the degradation of the new polymer BRS in the animal model is more uniform than a first generation BRS across 12 months. The novel BRS coating provided by the present invention can control degradation of the scaffold precisely.

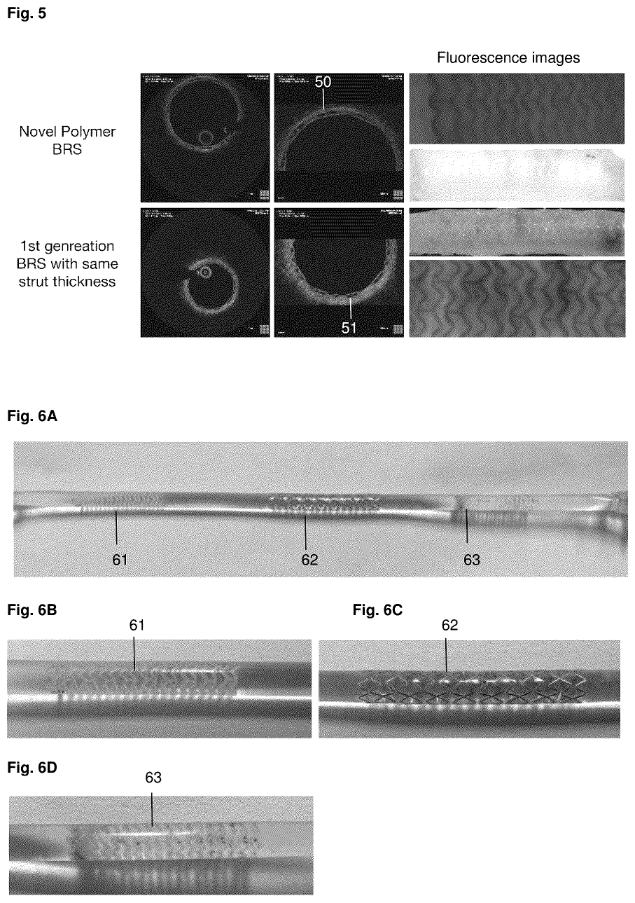

FIG. 5 demonstrates that the novel polymer BRS coating 50 demonstrates significantly faster endothelial repair in rabbit abdominal aorta compared to traditional drug-eluting BRS 51 with the same strut thickness (120 μm).

FIG. 6A-D show that the novel polymer coating BRS 61 in the semi-in vivo thrombosis model significantly improves its anti-thrombotic performance compared to the first-generation BRS 63, making it comparable to metal drug-eluting stent (DES) 62.

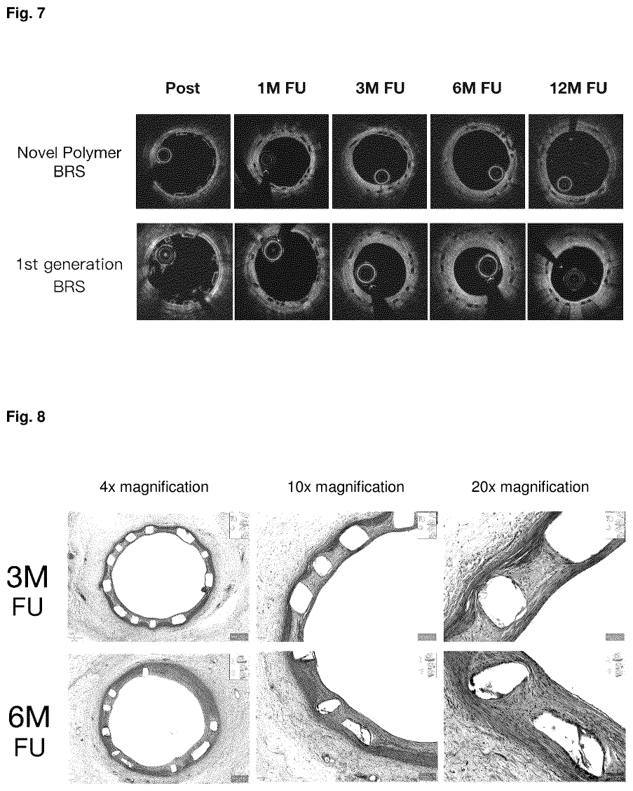

FIG. 7 displays sequential OCT follow-up images of the novel polymer coating BRS and first-generation BRS in porcine coronary arteries. It can be observed that the BRS vessel gradually expands, while there is no significant neointimal growth after one month. This indicates ideal control over the degradation process by the surface coating and prevents stent discontinuity.

FIG. 8 shows a typical histopathological section of the new BRS coating at 3 and 6 months follow up, revealing no significant inflammatory response around the stent. This indicates a mild and controllable degradation process.

### EXAMPLES

The following specific examples and test cases are provided to illustrate the implementation process and beneficial effects of the present invention, but do not limit the scope of the invention.

### Example 1

For example, this embodiment provides a vascular stent, including the same stent body, a degradation control layer with different proportions of hydrolytic polymer, the same hydrophobic layer, and the same drug layer.

The stent body is a mesh-like structure made of a copolymer of poly-L-lactide-co-caprolactone, with a number average molecular weight of 110,000.

The degradation control layer comprises a mixture of hydrophobic polymer and hydrolytic polymer. Its bioresorbable properties are controlled by proportion of both types of polymers.

The hydrophobic polymer is a copolymer of polyvinylidene fluoride-hexafluoropropylene, with a number average molecular weight of 200,000.

The hydrolytic polymer is a copolymer of polylactic acid and hydroxyethyl acrylate, with a number average molecular weight of 50,000.

The mass fractions of poly(lactic-co-glycolic acid)-co-poly(hydroxyethyl methacrylate) and the thicknesses of the degradation control layer 1 are shown in Table 1.

TABLE 1
Grouping of the degradation control layer 1 with
different poly(lactic-co-glycolic acid)-co-poly(hydroxyethyl
methacrylate) ratios and thicknesses.

| | Poly(lactic-co-glycolic | Thickness of | |
|---|---|---|---|
| Support | acid)-co-poly(hydroxyethyl | degradation | |
| group | methacrylate) ratio | control layer 1 | |
| Support A | 0.8% | 3000 | nm |
| Support B | 15.0% | 3000 | nm |
| Support C | 15.0% | 500 | nm |
| Support D | 30.0% | 500 | nm |

The hydrophobic layer comprises the hydrophobic polymer polyvinylidene fluoride-hexafluoropropylene with a number-average molecular weight of 380,000 and a thickness of 1500 nm.

The drug layer comprises the scaffold drug sirolimus and the drug carrier L-polylactic acid. The number-average molecular weight of L-polylactic acid is 80,000. The drug content of sirolimus on the unit length of the scaffold base is 10 ug/mm, and the ratio of the scaffold drug to the drug carrier is 1:3.

The preparation steps for the multi-functional composite coating high-polymer degradable stent in this embodiment are as follows:
1) The stent substrate is shaped into a mesh structure by laser cutting technology;
2) Different proportions of polyvinylidene fluoride-hexafluoropropylene copolymer and polylactic acid-glycolic acid copolymer are dissolved in acetone, and the mixture is fully coated on the surface of the stent substrate through the hollow ultrasound spraying process to form the degradation control layer 1 under different conditions for each stent group;
3) Polyvinylidene fluoride-hexafluoropropylene copolymer is dissolved in tetrahydrofuran, and then directionally sprayed on the outer surface of the degradable layer through the axial ultrasound spraying process. After drying, the hydrophobic layer is formed;
4) Sirolimus and L-polylactic acid are dissolved in tetrahydrofuran, and then fully coated on the outer surface of the degradation control layer and the hydrophobic layer 3 through the hollow ultrasound spraying process. After drying, the drug layer 4 is formed to complete the preparation of the multi-functional composite coating high-polymer degradable stent provided in this embodiment.

In addition, in this embodiment, the degradation control function of the support matrix of the multi-functional composite coated degradable polymer stent provided by the present invention is evaluated. The evaluation method follows the method described in “YY/T 0808-2010 In Vitro Pulsatile Durability Standard Test Method for Vascular Stents” to perform in vitro simulated degradation tests under near-physiological conditions. Samples of the stent base are taken at different degradation time points to test the number-average molecular weight. The faster the decrease in number-average molecular weight, the faster the degradation of the stent base.

The testing results are shown in FIG. 4A, indicating that:
a) Comparing the degradation curves of Stent A and B, reducing the proportion of hydrolytic high polymer in the degradation control layer provided by the present invention can effectively slow down the degradation rate of the support base.
b) Comparing the degradation curves of Stent B and C, reducing the thickness of the degradation control layer provided by the present invention can effectively accelerate the degradation rate of the support base.
c) Comparing the degradation curves of Stents C and D, significantly increasing the proportion of hydrolyic high polymer in the degradation control layer provided by the present invention can further accelerate the degradation rate of the support base.
   In addition, in this embodiment, stent B and 1st generation BRS are implanted into the coronary arteries of pigs. The number average molecular weight and weight average molecular weight of the stent are tested regularly to compare and evaluate the degradation control function of the coating provided by the present invention on the stent matrix. As shown in FIG. 4B, the coating provided by the present invention can effectively control the degradation process of the stent matrix, and has a smoother degradation curve compared to traditional BRS.

### Example 2

In this embodiment, a degradable polymer scaffold is provided, comprising a stent body, degradation control layer, hydrophobic layer, and drug layer.

The stent body is a mesh-like structure made of a poly-L-lactide-co-caprolactone copolymer with an average molecular weight of 110,000.

The degradation control layer is a blend of hydrophobic and hydrolytic high polymers, with a thickness of 5000 nm. The hydrophobic high polymer is a poly(vinylidene fluoride-co-hexafluoropropylene) copolymer with an average molecular weight of 200,000. The hydrolytic high polymer is a polylactic acid-glycolic acid copolymer with an average molecular weight of 50,000, with a mass fraction of 5% for the polylactic acid-glycolic acid copolymer.

The hydrophobic layer is made of a hydrophobic high polymer polyvinylidene fluoride-hexafluoropropylene copolymer with an average molecular weight of 380,000, with a thickness of 2000 nm.

The drug layer comprises scaffold drug sirolimus and drug carrier poly(lactic acid) with an average molecular weight of 80,000. The scaffold drug content on the scaffold base per unit length is 8 ug/mm, and the ratio of scaffold drug to drug carrier is 1:3. Alternatively, the drug layer comprises an antithrombotic agent.

The preparation steps for the biodegradable scaffold of this embodiment are as follows:
1) The scaffold base is processed into a mesh-like structure by laser cutting;
2) The polyvinylidene fluoride-hexafluoropropylene copolymer and polylactic-co-glycolic acid copolymer are dissolved in acetone and completely coated on the surface of the scaffold base using a hollow ultrasonic spraying process. After drying, the degradable layer under each scaffold group is formed;
3) The polyvinylidene fluoride-hexafluoropropylene copolymer is dissolved in tetrahydrofuran and directionally sprayed on the outer surface of the degradable layer using a through-axis ultrasonic spraying process. After drying, the hydrophobic layer is formed;
4) Sirolimus and left-handed polylactic acid are dissolved in tetrahydrofuran and completely coated on the outer surface of the degradable layer and hydrophobic layer using a hollow ultrasonic spraying process. After drying, the drug layer is formed, completing the preparation of the multifunctional composite coating biodegradable polymer scaffold provided by this embodiment.

As an example, the accelerated neointimal coverage rate characteristic of the vascular stent provided by the present invention is evaluated. The evaluation method utilizes the abdominal aorta of New Zealand white rabbits as the target vessel, where both the vascular stent provided in this embodiment and a traditional drug-coated bioresorbable stent are implanted. The neointimal coverage of both groups of stents is observed at two weeks and one month post-surgery. As shown in FIG. 5, the vascular stent provided in this embodiment demonstrates a significantly higher rate of neointimal coverage compared to the traditional drug-coated bioresorbable stent.

As an example, the antithrombotic performance of the vascular stent provided by the present invention is evaluated. The evaluation method is as follows: Both ends of a transparent compliant tube are connected to the femoral artery of a pig, allowing the arterial blood to flow through the compliant tube and back into the pig's vessels. The vascular stent provided in this embodiment, a traditional drug-coated metallic stent, and a traditional drug-coated bioresorbable stent are each implanted in the compliant tube. After one week, the compliant tube is removed and flushed with physiological saline to observe the thrombus attachment on each group of stents. As shown in FIG. 6, there is almost no thrombus formation on the vascular stent provided in this embodiment and the traditional drug-coated metallic stent, while significant thrombus formation is observed on the traditional drug-coated bioresorbable stent. These test results fully demonstrate that the vascular stent provided by the present invention possesses excellent antithrombotic performance.

### Example 3

This embodiment provides a multi-functional composite coating biodegradable polymer stent, comprising a stent body, a degradation control layer, a hydrophobic layer, and a drug layer.

The stent body is a pipe network structure made of poly-L-lactide-co-caprolactone copolymer with a number-average molecular weight of 110,000.

The degradation control layer is a blend of hydrophobic and hydrolytic polymers, with a thickness of 5000 nm.

The hydrophobic polymer is a copolymer of polyvinylidene fluoride-hexafluoropropylene with a number-average molecular weight of 200,000.

The hydrolytic polymer is a copolymer of lactide and glycolide with a number-average molecular weight of 50,000.

The mass fraction of the copolymer of lactide and glycolide is 0.5%.

The hydrophobic layer is made of hydrophobic polymer polyvinylidene fluoride-hexafluoropropylene copolymer with a number-average molecular weight of 380,000 and a thickness of 1500 nm.

The drug layer is made of the stent drug sirolimus and drug carrier poly-L-lactide, with a number-average molecular weight of 80,000 for poly-L-lactide. The stent drug content per unit length on the stent body is 14 ug/mm, and the ratio of stent drug to drug carrier is 1:4.

The preparation steps of the biodegradable stent according to this embodiment are as follows:
1) The stent base is processed into a mesh-like structure by laser cutting technology;
2) The fluorinated polymer polyvinylidene fluoride-hexafluoropropylene copolymer and the hydrolytic polymer polylactic acid-co-glycolic acid copolymer are dissolved in acetone and completely sprayed onto the surface of the stent base through a hollow ultrasonic spraying process. After drying, the degradation control layer under various stent grouping conditions is formed;
3) Polyvinylidene fluoride-hexafluoropropylene copolymer is dissolved in tetrahydrofuran, and directed spraying is performed on the outer surface of the degradation control layer through a cross-axis ultrasonic spraying process. After drying, the hydrophobic layer is formed;
4) Sirolimus and left-handed polylactic acid as the drug carrier are dissolved in tetrahydrofuran, and complete spraying is performed on the outer surface of the degradable layer and the hydrophobic layer through a hollow ultrasonic spraying process. After drying, the drug layer is formed, completing the preparation of the multi-functional composite coating biodegradable polymer stent according to this embodiment.

This embodiment evaluates the functions of the multifunctional composite coating provided, which include controlling the degradation process of the biodegradable polymer stent, preventing thrombus formation, inhibiting neointimal hyperplasia, and promoting endothelialisation. The evaluation method uses the coronary artery of domestic pigs as the implanted blood vessel and implants the stent provided in this embodiment. OCT follow-up evaluations are performed 1 month, 3 month, 6 month and 1 year. Pathological evaluations were performed at 3 and 6 months.

FIG. 7 displays sequential OCT follow-up images of the novel polymer coating BRS and first-generation BRS in porcine coronary arteries. It can be observed that the BRS vessel gradually expands, while there is no significant neointimal growth after one month. This indicates ideal control over the degradation process by the surface coating and prevents stent discontinuity.

FIG. 8 shows a typical histopathological section of the new BRS coating at 3 and 6 months follow up, revealing no significant inflammatory response around the stent. This indicates a mild and controllable degradation process.

The evaluation results shown in FIGS. 7 and 8 indicate that the multifunctional composite coating provided by the present invention effectively controls the degradation process of the stent substrate. Specifically, it regulates the hydrolysis rate of the polymer substrate during the early stages of degradation, preventing rapid degradation that could lead to loss of support, inflammatory reactions, thrombus formation, and neointimal hyperplasia. However, in the later stages of degradation, it does not impede the exchange and absorption of hydrolysis by-products, and the overall degradation time is not prolonged.

### Conclusion

The field of vascular stents has a large patient base, but the late adverse event rate of traditional metal stents remains high. Bioresorbable stents have broad market prospects, and many companies and research institutions worldwide are investing significant resources and time in related research. However, clinical results have not been satisfactory, and the complexity of the actual clinical environment is the main reason. The development direction of “complete degradation” faces great difficulties.

From the current clinical and basic research conclusions, the core advantage of bioresorbable stents lies in the restoration of vascular function. Complete absence is no longer the core requirement of degradable stents. Therefore, a durable coating with good biocompatibility can still achieve the restoration of vascular function without affecting the hydrolysis of the stent substrate material. At the same time, minimal residue of the coating material will not cause significant long-term inflammation in blood vessels, and can avoid long-term stimulation of foreign bodies on the vessel wall and stent structural detachment, thereby reducing the incidence of late adverse events.

In summary, the field of bioresorbable stents has enormous market potential and a long-felt want. This invention provides a new direction for product development from the perspective of clinical application, effectively solves clinical pain points, expands the range of indications and lesion locations, has a simple process, low cost, and has high commercial value.

1. A coating for a bioresorbable scaffold, comprising a degradation control layer comprising a durable hydrophobic polymer and a biodegradable hydrolytic polymer.

2. The coating of claim 1, wherein the hydrophobic polymer comprises at least one polymer selected from a group consisting of: acrylics, amides, imides, carbonates, dienes, esters, ethers, fluorocarbons, olefins, styrenes vinyl acetals, vinyl and vinylidene chlorides, vinyl esters, vinyl ethers, vinyl ketones, vinylpyridine, vinypyrrolidone, poly(vinylidene chloride), polyvinylidene fluoride (PVDF), polyvinylidene fluoride-co-hexafluoropropylene (PVDF-HFP), styrene-butadiene-styrene copolymer (SBS), polyurethane (PU), polymethyl methacrylate (PMMA), and polybutyl methacrylate (PBMA), poly(N-vinylpyrrolidone), poly(vinylpyridine), poly(vinylpyrrolidone), polydimethylsiloxane (PDMS), poly-xylene (PX), and combinations thereof.

3. The coating of claim 1, wherein the hydrolytic polymer comprises at least one polymer selected from the group comprising: polylactic acid (PLA), poly-L-lactic acid (PLLA), poly-D-lactic acid (PDLA), poly-DL-lactic acid (PDLLA), poly(lactic-co-glycolic acid) (PLGA), poly(lactic-co-glycolic) acid, Dextron, nylon-2-nylon-6, PHBV polymer (Poly(3-hydroxybutyrate-co-3-hydroxyvalerate)), poly-L-proline ester-hexyl lactate copolymer, polyglycolic acid (PGA), polyhydroxy butyrate (PHB), polycaprolactone, polyglycolide, polylactide, chitosan, hyaluronic acid, poly(2-hydroxyethyl-methacrylate), poly(L-lactide), polyethylene glycol, chitosan, and combinations thereof.

4. The coating of claim 1, wherein thickness of the degradation control layer is in the range of 200 nm and 20,000 nm.

5. The coating of claim 1, wherein the hydrolytic polymer is in the range between 0% to 50% by mass fraction.

6. The coating of claim 1, further comprising a drug layer comprising a drug.

7. The coating of claim 6, wherein the drug comprises an immunosuppressant.

8. The coating of claim 7, wherein the immunosuppressant comprises at least one compound selected from the group comprising sirolimus, everolimus, zotarolimus, tacrolimus, paclitaxel, and combinations thereof.

9. The coating of claim 6, wherein the drug is deposited in a layer of thickness in the range of 500 nm and 10,000 nm.

10. The coating of claim 6, wherein the drug amount per unit length of the coating is in the range of 3 μg/mm to 25 μg/mm.

11. The coating of claim 6, wherein a ratio of the drug to a drug carrier is in the range of 3:1 to 1:10.

12. The coating of claim 1, wherein thickness of the hydrophobic layer is in the range of 200 nm to 10,000 um.

13. A bioresorbable scaffold comprising a stent body and a coating of claim 1.

14. The bioresorbable scaffold of claim 13, wherein the stent body comprises at least one hydrolytic polymer comprising polylactic acid, poly-L-lactic acid, poly-caprolactone, poly-L-proline ester-hexyl lactate copolymer and/or polyglycolic acid.

15. A method of coating a stent body to make a bioresorbable scaffold, comprising:
depositing a degradation control layer on the stent body, wherein the degradation control layer comprises a durable hydrophobic polymer and a biodegradable hydrolytic polymer.

16. The method of claim 15, wherein the hydrophobic layer forms a discontinuous layer around the degradable layer.

17. The method of claim 15, further comprising depositing a drug layer comprising a medicament dissolved in a solvent.

18. The method of claim 15, wherein the depositing comprises ultrasonic spray coating.

19. The method of claim 17, wherein the degradation control layer and/or the drug layer comprises a polymer is dissolved in a volatile organic solvent at a concentration between 0.5 mg/ml and 50 mg/ml by weight prior to depositing.

20. The method of claim 15, wherein the stent body comprises at least one hydrolytic polymer comprising polylactic acid, poly-L-lactic acid, poly-caprolactone, poly-L-proline ester-hexyl lactate copolymer and/or polyglycolic acid.
